(19) United States
(12) Patent Application Publication
(10) Pub. No.: US 2021/0292827 A1
(43) Pub. Date: Sep. 23, 2021

(54) IMPROVED FORENSIC METHODS AND PRODUCTS FOR EXTRACTING AND AMPLIFYING TRACE DNA FOR DNA SEQUENCING

(71) Applicant: THE SECRETARY OF STATE FOR DEFENCE, SALISBURY, WILTSHIRE (GB)

(72) Inventor: CARL NICHOLAS MAYERS, (US)

(21) Appl. No.: 17/250,733

(22) PCT Filed: Sep. 6, 2019
(86) PCT No.: PCT/GB2019/000126

§ 371 Date: Feb. 26, 2021

(30) Foreign Application Priority Data
Sep. 10, 2018 (GB) 1814676.1

(51) Int. Cl. C12Q 1/6848; C12Q 1/6806; C07H 21/04

(52) U.S. Cl. CPC C12Q 1/6848; C07H 21/04; C12Q 1/6806

(57) ABSTRACT

The present invention provides a synthetic nucleic acid for use in methods for interrogating samples suspected of comprising trace DNA. The present invention is especially concerned with forensic analysis of trace DNA, and especially methods and products for achieving collection, extraction and amplification of trace DNA samples capable of limiting the loss of the trace DNA, whilst improving the amplification and the subsequent likelihood of achieving positive sequencing of the trace DNA.

The present invention is concerned with improved methods and products for forensic analysis of trace DNA, and especially methods and products for achieving collection, extraction and amplification of trace DNA samples capable of limiting the loss of the trace DNA, whilst improving the amplification and the subsequent likelihood of achieving positive sequencing of the trace DNA.

DNA evidence sample processing, such as for trace or touch DNA, typically involves DNA collection, extraction, and amplification, followed by DNA sequencing; however, DNA loss can occur at all stages of the process, especially the collection and extraction, but also potentially during the amplification stage, which is not ideal for forensic evidence containing low levels of DNA (i.e. trace or touch DNA samples).

Factors that affect DNA collection and extraction efficiency include non-specific absorption of DNA to the collection/extraction tubes used in the analysis, the number of times material is transferred from one tube to another, and the number of washing steps. Clearly the capacity of DNA to absorb/irreversibly bind to plastic consumables and extraction matrices can be critical to whether trace DNA is eventually detected and identified. For example, silica matrices (silica based tubes) are capable of irreversibly binding nucleic acid, and so may contribute to sample loss. Sample loss through non-specific absorption can however be avoided or mitigated if a carrier or blocker substance is present in the collection media/buffer and/or extraction media/buffer, such as a carrier/blocker polynucleotide, such as a different RNA or DNA in the extraction buffer.

Thus, when trace/touch DNA is collected from a crime scene or exhibit, carrier DNA or RNA may be added to the extract to help stabilise the trace DNA, and critically prevent or mitigate loss of the trace DNA through having a much higher concentration of the carrier DNA/RNA. This may then prevent against loss of DNA by adsorption to surfaces, and aid the collection, extraction, and subsequently amplification, of DNA by increasing the DNA concentration available. A commonly used carrier DNA is salmon sperm DNA.

The use of carrier DNA/RNA can however provide significant problems itself, especially when the trace DNA is amplified and subsequently sequenced as the majority of the DNA sequence obtained is then derived from the carrier DNA (i.e. salmon genomic DNA), rather than the desired trace human or metagenomic DNA.

There is consequently a requirement to provide new and alternative carrier/blocker substances which prevent loss of DNA by adsorption to surfaces, but which avoid the trace DNA being swamped by any carrier DNA that may be present.

The present invention thus generally aims to provide new and alternative carrier/blocker substances which can be used during the collection, extraction and amplification of trace DNA, which avoid loss of the trace DNA by adsorption to surfaces, but which also avoid the trace DNA being swapped by carrier DNA.

Thus, in a first aspect, the present invention provides a synthetic nucleic acid comprising multiple restriction enzyme recognition sites, wherein at least 20% of the nucleotides in the synthetic nucleic acid are within a restriction enzyme recognition site.

The Applicant has designed a synthetic nucleic acid that comprises a high density of restriction enzyme recognition sites, which high density is provided by at least 20% of the nucleotides in the synthetic nucleic acid being within a restriction enzyme recognition site. Such a nucleic acid has been designed for use as a carrier or blocker substance in the collection, extraction and amplification of trace DNA, preventing loss of DNA through adsorption, but critically which can, for example, be digested during amplification by including a restriction enzyme or multiple restriction enzymes in the amplification buffer, in order that the amplified trace DNA is not swamped by carrier DNA in the final analysis, since the carrier DNA has been digested into small nucleic acid fragments (for example oligonucleotides).

The high density of restriction enzyme recognition sites in the synthetic nucleic acid may be provided by at least 25%, at least 30%, at least 50%, or at least 75% of the nucleotides being within a restriction enzyme recognition site. The synthetic nucleic acid may however be comprised solely of restriction enzyme recognition sites, and thus 100% of the nucleotides in the synthetic nucleic acid may be within such a site.

Preferably the numerous restriction enzyme recognition sites are the same restriction enzyme recognition site, and thus are specific to a single restriction enzyme. The single restriction enzyme recognition site is preferably one that is rare in native DNA nucleic acid sequences.

In one embodiment the restriction enzyme recognition site is a site that is naturally particularly rare in a native DNA nucleic acid sequence. Eight (8) base pair restriction enzyme recognition sites, of which a few exist, are known to be rare in native DNA nucleic acid sequences, and thus in one embodiment the restriction enzyme recognition site is one which consists of a specific eight base pair sequence. The restriction enzyme site may in particular be that for the restriction enzyme from Streptomyces fimbriatus, Sfil, which recognises and cleaves at 5′-GGCCNNNNNGGCC-3′ sites, wherein N is any nucleotide.

The synthetic nucleic acid may consist solely of multiple successive/repetitive recognition sites recognised by a single restriction enzyme, wherein the enzyme may in particular be the Sfil restriction enzyme.

The synthetic nucleic acid of the first aspect has been designed and engineered such that it can be used during the collection, extraction and amplification of trace DNA to prevent or mitigate loss of the trace DNA through non-specific absorption to materials used in the process, such as collection, extraction or amplification tubes, but which is also able to be digested by the restriction enzyme specific to the recognition site either during or following the amplification step of the process, so that the synthetic nucleic acid is degraded, and thus only the amplified trace DNA remains for sequencing to identify its sequence.

Thus, in a second aspect, the present invention provides a method for collecting a sample suspected of comprising trace DNA, said method comprising collecting the sample into a buffer comprising the synthetic nucleic acid of the first aspect.

In a third aspect, the present invention provides a method for interrogating a sample suspected of comprising trace DNA, said method comprising collecting the sample, extracting the trace DNA, and amplifying the trace DNA in the presence of the synthetic nucleic acid of the first aspect, wherein amplifying the trace DNA is undertaken in the presence of a restriction enzyme or multiple restriction enzymes which recognise(s) and cleave(s) at the restriction enzyme recognition site(s) incorporated in the synthetic nucleic acid, or alternatively the restriction enzyme(s) is/are added following amplification of the trace DNA.

The invention of the second and third aspect uses a synthetic carrier nucleic acid that has been engineered to contain a high density of restriction enzyme recognition sites, in particular a high density of a single restriction enzyme recognition site, and preferably a restriction enzyme recognition site rarely found in native DNA nucleic acid sequences. The recognition site may be that recognised by the restriction enzyme Sfil, in which case the restriction enzyme would be Sfil, or a restriction enzyme recognising the same recognition site. This restriction enzyme recognition site was selected because it is extremely rare in genomic DNA (it has an eight base pair motif, separated by 5 non-specific base pairs).

Advantageously, the Sfil enzyme is heat stable at elevated temperature, and importantly is not denatured by the PCR process, and is active in a wide range of commonly used amplification buffers. The efficiency of the Sfil enzyme is also increased as the concentration of recognition sites increases, providing an exponential increase in destruction of the synthetic fragment as the reaction progresses.

### EXAMPLES

A synthetic nucleic acid comprising numerous restriction enzyme recognition sites can be synthesised using methods known in the art. A synthetic nucleic acid comprising numerous, repetitive/successive Sfil recognition sites was synthesised. Since the recognition site comprises the same 4 nucleotide sequence of GGCC separated by five non-specific nucleotides, then a synthetic sequence of 900 bases long would contain 100 possible restriction enzyme cleavage sites

The synthetic nucleic acid can then be added to a trace DNA sample either in the collection or extraction or amplification buffer, to protect the trace DNA from any loss through non-specific absorption. Once the trace DNA is being prepared for sequencing, the Sfil enzyme may be added before, during or after the amplification reactions used for sequencing library construction (e.g. PCR). The Sfil enzyme is active in the amplification buffer, and critically is not inhibited by the PCR reaction, or at any of the temperatures required for amplification. As the amplification reaction proceeds, all trace DNA is amplified, while the synthetic nucleic acid is efficiently degraded. This thus results in an amplified library of trace DNA ready for sequencing that contains no contaminating DNA.

The synthetic nucleic acid comprises an extremely high density of a rare restriction enzyme recognition site that can be cut using a restriction enzyme that can be added as a supplement to amplification reactions such as the polymerase chain reaction. This prevents any of the synthetic carrier from being amplified, while maintaining its carrier function. Furthermore, the restriction enzyme in this example (Sfil) is catalysed by cis- and trans-restriction sites with the high density of restriction enzyme sites greatly increasing the efficiency of digestion of the synthetic carrier DNA.

The specific sequence of the synthetic carrier DNA is optimised for maximum efficiency and density of restriction sites.

This new approach and method allows all the benefits of carrier-DNA to be achieved to protect the trace DNA and improve efficiency, but in addition allows the carrier DNA to be specifically degraded when required without additional steps in the process. It can in particular allow for more sensitive sequencing of trace DNA from extremely low levels of trace human and metagenomic DNA.

1. A synthetic nucleic acid comprising multiple restriction enzyme recognition sites, wherein at least 20% of the nucleotides in the synthetic nucleic acid are within a restriction enzyme recognition site, and wherein the restriction enzyme recognition sites are the same recognition site specific to a single restriction enzyme.

2. A synthetic nucleic acid according to claim 2, wherein at least 50′% of the nucleotides in the synthetic nucleic acid are within a restriction enzyme recognition site.

3. A synthetic nucleic acid according to claim 1, wherein the restriction enzyme recognition site is specific to the restriction enzyme from Streptomyces fimbriatus (Sfil).

4. A synthetic nucleic acid according to claim 1, wherein the recognition site has the sequence 5″--GGCCNNNNNGElCC-3′.

5. A synthetic nucleic acid according to claim 1 consisting of multiple repetitive restriction enzyme recognition sites.

6. A method for collecting a sample suspected of comprising trace DNA, said method comprising collecting the sample into a buffer comprising the synthetic nucleic acid according to claim 1.

7. A method for interrogating; a sample suspected of comprising trace DNA, said method comprising collecting the sample, extracting the trace DNA, and amplifying the trace DNA in the presence of the synthetic nucleic acid according to claim 1, wherein amplifying the trace DNA is undertaken in the presence of a restriction enzyme which recognizes and cleaves at the restriction enzyme recognition sites incorporated in the synthetic nucleic acid, or alternatively the restriction enzyme is added following amplification of the trace DNA.

8. A method according to claim 7, wherein the restriction enzyme site is specific to the restriction enzyme from Streptomyces fimbriatus (Sfil), and the restriction enzyme is from Streptomyces fimbriatus.
